(19) United States
(12) Patent Application Publication
(10) Pub. No.: US 2010/0291054 A1
(43) Pub. Date: Nov. 18, 2010

(54) NOVEL REGULATORY T CELLS AND USES THEREOF

(75) Inventors: Xin Xiao Zheng, Pittsburgh, PA (US); Dong Zhang, Pittsburgh, PA (US)

Correspondence Address: CLARK & ELBING LLP, 101 FEDERAL STREET, BOSTON, MA, 02110, US

(73) Assignee: BETH ISRAEL DEACONESS MEDICAL CENTER, Boston, MA (US)

(21) Appl. No.: 12/516,891

(22) PCT Filed: Nov. 28, 2007
(86) PCT No.: PCT/US07/24505

§ 371 Date: Aug. 3, 2010

Related U.S. Application Data
(60) Provisional application No. 60/861,770, filed on Nov. 29, 2006.

(51) Int. Cl. A61K 35/12; C12N 5/0783; A61P 37/02

(52) U.S. Cl. 424/93.71; 435/372.3

(57) ABSTRACT

The invention provides isolated regulatory T cells and methods of obtaining regulatory T cells. The invention also provides methods for inhibiting an antigen-specific immune response (e.g., graft rejection, an autoimmune disorder, graft versus host disease, a response to a tumor cell, a response to an infection, and a response to an allergen) in a subject requiring administering an isolated regulatory T cell to the subject. The invention further provides methods for treating or modulating an antigen-specific immune response in a subject requiring administering a regulatory T cell to the subject.

### BACKGROUND OF THE INVENTION

This invention relates to the field of antigen specific immunity, the development and modulation thereof.

The immune system is a homeostatic organization that must regulate itself to avert insufficient immunity, suppress excessive responses, and prevent auto-reactive responses. This finite regulation is mediated, in part, by a group of T lymphocytes identified as regulatory T cells. Accumulating evidence in supporting the existence of more than one population of regulatory T-cells that are engaged in the maintenance of peripheral tolerance. These different regulatory populations function in different ways and some are naturally produced and other are locally induced as a result of immune responses. Although numbers of studies reported that in the peripheral lymphoid tissues of normal mice and humans 1-5% of total lymphocytes are αβ-TCR⁺ DN T cells, and an age-related accumulation of αβ-TCR⁺ DN T cells in MRL/Mpj-lpr/lpr mice, which have a mutant Fas gene and massive lymphadenopathy, the origin of peripheral DN T cells is still unclear. The heterogeneity in markers expressed by different DN T cells suggests that several maturation/differentiation pathways may exist. In murine models several studies have demonstrated that DN αβ TCR⁺ T cells can be derived directly from CD8⁺ T cells. Other studies suggest that DN αβ TCR⁺ cells are derived extrathymically from organs such as bone marrow.

As regulatory T cells play important role in maintaining peripheral tolerance, the therapeutic potential for transfer of regulatory T cells is of interest for use in autoimmune disease and transplantation. However, current technology using regulatory T cell population(s) as a potential cell-based therapeutic for the treatment of immune-mediated disorders has met with limited success because lack of precise cell markers, lack of antigen specificity, and the lack of a feasible source of regulatory cells.

A means of isolation, and ex vivo identification and propagation of regulatory T cells is needed to improve current cell-based therapeutic regimens for the treatment of immune-mediated disorders.

### SUMMARY OF THE INVENTION

The invention features a unique pathway for differentiating a regulatory T cell, said cell having the phenotype CD4⁻, CD8⁻, CD3⁺ (double negative T cells, DN T cells) and expressing at least one of the markers CD44⁺, CD69⁺, or CD28⁺.

The invention also features a method for obtaining a regulatory T cell with the phenotype CD4⁻, CD8⁻, said method comprising of isolating a CD4⁺, CD8⁻ cell from a sample; culturing said CD4⁺, CD8⁻ cell with antigen and at least one of IL-2, or IL-15; isolating said CD4⁻, CD8⁻ cell; wherein said isolated CD4⁻, CD8⁻ cell has the characteristics of suppressing an antigen-specific immune response to said antigen in a subject. The isolated CD4⁺, CD8⁻ precursor cell can have the phenotype CD25⁺ or CD25⁻, and the converted CD4⁻, CD8⁻ cell from either precursor is Foxp3⁻. In another preferred embodiment, said DN regulatory T cell obtains a CD4⁻ phenotype as the result of CD4 gene silencing.

In another feature of the invention, said regulatory T cell also expresses at least one of the markers CD3⁺, CD25⁺, TCR β⁺, but not NK1.1⁻, and is Foxp3⁻. Preferably, said regulatory T cell also expresses low levels of IL-2, IL-4, IFN-γ, CTLA-4, TGF-β, and high levels of perforin and granzyme B.

In another embodiment of the invention, said CD4⁻, CD8⁻ cell suppresses at least one of proliferation, or activation of an antigen-specific responder T cell. This includes an embodiment where said regulatory cell is more effective at suppressing antigen-specific proliferation of naïve CD4⁺, CD25⁻ responder T cells than at suppressing antigen-nonspecific proliferation of naïve CD4⁺, CD25⁻ responder T-cells.

In another embodiment of the invention, said regulatory T cell is hypo-responsive when challenged with antigen, and responsiveness can be restored by at least one of IL-2, or IL-15.

The invention also features a method for obtaining a CD4⁻, CD8⁻ regulatory T cell that expresses at least one of the following markers CD3⁺, TCR β⁺, CD44⁺, CD69⁺, or CD28⁺, and the proteins perforin and granzyme B.

The invention also features a method for obtaining a CD4⁻, CD8⁻ regulatory T cell by at least 4 rounds of antigen stimulated proliferation. The said CD4⁻, CD8⁻ regulatory T cell suppresses at least one of proliferation, or activation of an antigen-specific responder T cell. The responder T cells may be CD4⁺, CD25⁻ or CD8⁺.

Another feature of this invention includes obtaining a CD4⁻, CD8⁻ regulatory T cell that is antigen-specific, wherein said antigen is an auto-, allo-, or xenoantigen. Preferably, this antigen is present on CD3⁻ mature bone marrow dendritic cells, B cells or other antigen presenting cells.

The invention also features a method of isolating said CD4⁻, CD8⁻ regulator T cell by selection of said cell expressing the cell surface marker CD3 and not expressing the cell surface marker CD4. If desired, the method of isolating said CD4⁻, CD8⁻ regulator T cell is done using at least one of an enrichment column, or cell sorting. In another preferable embodiment of this invention, said isolated CD4⁻, CD8⁻ regulator T cell is expanded by at least one of IL-2, or IL-15.

The invention also features a method for inhibiting an antigen specific immune response in a subject in need thereof, wherein said method comprising of administering said CD4⁻, CD8⁻ regulatory T cell.

The invention also features a method for treating an antigen specific immune response in a subject in need thereof, wherein said method comprising of administering said CD4⁻, CD8⁻ regulatory T cell.

This invention also features a method for modulating an antigen-specific immune response in a subject in need thereof, wherein said method comprising of administering said CD4⁻, CD8⁻ regulatory T cell.

In the forgoing aspects of inhibiting, treating or modulating an antigen-specific response, said antigen-specific immune response may be graft rejection, an autoimmune disorder, graft versus host disease (GVHD), a response to a tumor cell, a response to an infection, or a response to an allergen. The method of said inhibition, treatment or modulation includes augmenting activation induced cell death (AICD) of naïve or activated responder T cells, in a patient in need thereof. The said responder T cells may be CD4⁺, CD25⁻ or CD8⁺. Preferably, the method of said AICD is by apoptosis of said responder cells, wherein said AICD is partially dependent on perforin or granzyme B.

### BRIEF DESCRIPTION OF THE DRAWINGS

FIG. 1A is a histogram of CFSE labeled T cell proliferation induced by allogeneic mature DC and cytokines. CD4⁺CD25⁻ (Teff) and CD4⁺CD25⁺ (Treg) cells from C57BL/6 mice were stimulated with mature DBA/2 DC plus rIL-2 or rIL-15 for 5 days. CD3⁺ T cells were gated and subjected to CFSE analysis.

FIG. 1B is a schematic illustration demonstrating the conversion of CD4⁻ cells from C57BL/6 CD4⁺ T cells via mature DBA/2 DC stimulation. Numbers beside outlined areas indicate the percentage of cells in the designated gate.

FIG. 1C is a schematic illustration showing that CD4⁻ cells appeared only after several cycles of cell proliferation induced by allogeneic mature DC.

FIG. 1D is a histogram and a schematic illustration demonstrating that at an equivalent concentration to support alloantigen triggered CD4⁺ T cell proliferation, rIL-2 and rIL-15 exert similar potency to enhance the conversion of CD4⁺ into CD4⁻ T cells.

FIG. 2A is a schematic illustration demonstrating the conversion of CD4⁻ cells from C57BL/6 (CD45.1) CD4⁺ T cells via mitomycin C treated DBA/2 allogeneic APC stimulation in vitro.

FIG. 2B is a schematic illustration demonstrating the conversion of CD4⁻ cells converted from C57BL/6 (CD45.1) CD4⁺ T cells via syngeneic mature DC stimulation in vitro.

FIG. 2C is a schematic illustration demonstrating the conversion of CD4⁻ cells from CD4⁺ precursors in vivo. CFSE labeled allogeneic C57BL/6 (CD45.1) CD4⁺ T cells were transferred to B6D2F1 mice by i.v. injection. Flow cytometry is of CD4⁻ cells converted from C57BL/6 (CD45.1) CD4⁺ T cells harvested from spleens and lymph nodes of B6D2F1 mice on day 3.

FIG. 2D is a schematic illustration demonstrating the conversion and maintenance of CD4⁻ cells from CD4⁺ precursors in vivo. CFSE labeled C57BL/6 (CD45.1) CD4⁺ T cells were transferred to syngeneic Rag KO mice by i.v. injection. Flow cytometry is of CD4⁻ cells converted from C57BL/6 (CD45.1) CD4⁺ T cells harvested from spleens and lymph nodes of syngeneic Rag KO mice on day 7.

FIG. 3A is a histogram showing staining of the converted CD4⁻ T cells with antibodies to indicated cell surface markers.

FIG. 3B is a schematic illustration demonstrating the relative expression of CD4 and CD8 genes by real-time PCR on different cell populations. Results shown here represent three independent experiments.

FIG. 3C is a histogram and schematic illustration demonstrating that most of the DN T cells are Annexin V⁻ although the majority of activated CD4⁺ T cells are Annexin V⁺.

FIG. 3D is a schematic illustration demonstrating that converted CD4⁻ cells express a unique expression profile. CD4⁺CD25⁻ (Teff) and CD4⁺CD25⁺ (Treg) cells from Foxp3-GFP knockin C57BL/6 mice were stimulated with mature DBA/2 DC alone or plus rIL-15 for 6 days. The DN T cells are GFP⁻ (Foxp3).

FIG. 3E is a schematic illustration showing the relative expression of indicated genes as determined by real-time PCR on different cell populations. Results shown here represent four independent experiments.
* Teff DN: DN T-cells converted from CD4⁺CD25⁻ T cells.
** Treg DN: DN T-cells converted from CD4⁺CD25⁺ T regs.

FIG. 4A is a schematic illustration showing the activation profile of DN T cells and CD4⁺ T cells isolated from primary MLR stimulated by DBA/2 mature DC plus IL-15, and re-stimulated by mature DBA/2 DC plus indicated cytokines for 4 days. Proliferation was determined by tritiated thymidine incorporation ([³H] TdR) incorporation and shown as means of three independent experiments.

FIG. 4B is a schematic illustration demonstrating that DN T-cells remain DN phenotype 4 days after re-stimulation with mature DC with or without IL-2 or IL-15.

FIG. 4C is a histogram demonstrating C57BL/6 DN T cells induced by mature DBA/2 DC potently suppress CFSE labeled C57BL/6 (CD45.1) Teff proliferation triggered by same allo-antigens (mature DBA/2 DC).

FIG. 4D is a histogram demonstrating that C57BL/6 DN T cells induced by mature DBA/2 DC suppress CFSE labeled C57BL/6 (CD45.1) Teff proliferation triggered by third party allo-antigens (mature C3H DC) at lower efficacy.

FIG. 5A is a histogram demonstrating Annexin V staining of C57BL/6 Teff stimulated by mature DBA/2 DC. C57BL/6 DN T cells greatly increased the Annexin V⁺ population in proliferating C57BL/6 Teff.

FIG. 5B is a histogram demonstrating a role for perforin in the suppression of antigen specific responses. CFSE labeled C57BL/6 (CD45.1) Teff cells were stimulated by mature DBA/2 DC. The suppressor function of C57BL/6 DN T cells converted from wild type and perforin knockout mice was compared. Results shown here represent three independent experiments.

FIG. 5C is a schematic illustration showing the DN T cell mediated suppression of Teff cell proliferation was attenuated in the absence of perforin. Results shown here are means of three independent experiments.

FIG. 5D is a schematic illustration showing that the perforin-mediated suppression of Teff cell proliferation was likely apoptosis of the Teff cells. Annexin V staining of C57BL/6 Teff stimulated by mature DBA/2 DC. DN T cells converted from wild type, but not perforin KO mice, greatly increased the Annexin V⁺ population in proliferating C57BL/6 Teff.

FIG. 5E is a schematic illustration showing that DN T cell mediated suppression of Teff cell proliferation was attenuated by granzyme B blockage. Results shown here are means of three independent experiments.

FIG. 5F is a schematic illustration of CFSE labeled B6 Teff cells that were stimulated by mature DBA/2 DC. The suppressor function of B6 DN T cells was tested in the presence of granzyme B or control antibodies.

FIG. 6A is a schematic illustration showing that DN T cells suppress naïve T effector triggered skin allograft rejection in an alloantigen specific manner. The rejection of skin graft from DBA/2 or C3H mice transplanted to C57BL/6 RAG (⁻/⁻) mice was induced by adoptive transfer of naïve C57BL/6 Teff cells. Co-transfer of C57BL/6 DN T cells suppressed the rejection more efficiently in mice received DBA/2 grafts. Statistical analyses were performed using a Logrank test.

FIG. 6B is a schematic illustration showing that DN T-cells significantly prolonged MHC mismatched islet allograft survival in an alloantigen specific manner in immune competent recipients. Administration of 13×10⁶ DN T-cells significantly prolonged alloantigen specific DBA/2, but not third party C3H, islet allograft survival. Statistical analyses were performed using a Logrank test.

FIG. 7A is a schematic diagram showing that DN T cells protect NOD/SCID mice from autoimmune diabetes induced by diabetogenic T cells. Diabetes in NOD/SCID mice was induced by T cells from diabetic NOD mice. Co-injection of NOD DN T cells significantly protected the mice from diabetes. Statistical analyses were performed using a Logrank test.

FIG. 7B is a schematic diagram showing that islet GAD65 antigen specific DN T cells were more potent than antigen nonspecific DN T cells in blocking autoimmune diabetes in new onset diabetic NOD mice. The new onset diabetic NOD mice were transferred with GAD65 specific or nonspecific DN T cells. Statistical analysis were performed using a Logrank test.

FIG. 8 is a model demonstrating the intrinsic homeostatic mechanisms that occur during the initial antigen-induced activation of CD4⁺ T cells control the magnitude and class of immune responses, including the emergence of T_{H}1, T_{H}2, T_{H}17 effectors and CD4⁺CD25⁺Foxp3⁺, Trl, and CD4⁺ converted DN regulatory cells. The dichotomy of T_{H}1 and T_{H}2 T cell subsets, the reciprocal differentiation of Treg and T_{H}17 effectors, and the subsequent activation induced cell death elucidate how the intrinsic homeostatic mechanisms control the magnitude and class of immune responses to infectious organisms and tissue inflammation. A new pathway of differentiating previously unidentified DN regulatory T cells represents a negative feedback mechanism that regulates the magnitude of immune responses.

### DETAILED DESCRIPTION OF THE INVENTION

Different regulatory populations of T cells function in different ways, and some are naturally produced and other are locally induced as a result of immune responses. By monitoring the CD4 expression during CD4⁺ T cell proliferation and differentiation, we identified a new pathway to differentiate a double negative (DN) regulatory T-cell subset. The invention describes an isolated regulatory T cell, said cell having the unique phenotype CD4⁻, CD8⁻, and expressing at least one of the markers CD44⁺, CD69⁺, or CD28⁺, but not NK1.1. In a preferable embodiment of this invention, the CD4⁻, CD8⁻ regulatory T cell is Foxp3⁻.

The invention further describes CD4⁻, CD8⁻ regulatory T cell which expresses a unique set of surface markers and gene profile that differ from previously identified regulatory T-cells. Preferably, said CD4⁻, CD8⁻ regulatory T cell also expresses low levels of IL-2, IL-4, IFN-γ, CTLA-4, TGF-β, and high levels of perforin and granzyme B.

In a preferred embodiment of the invention, said CD4⁻, CD8⁻ regulatory T cell is more effective at suppressing antigen-specific proliferation of naïve CD4⁺, CD25⁻ T-cells than said cell is at suppressing naïve and activated CD4⁺, CD25⁺. The invention further describes a method for obtaining a regulatory T cell with the phenotype CD4⁻, CD8⁻, said method comprising of: isolating a CD4⁺, CD8⁻ cell from a sample; culturing said CD4⁺, CD8⁻ cell with antigen and at least one of the cytokines IL-2, or IL-15; and isolating a converted CD4⁻, CD8⁻ regulatory T cell. It is desirable that the said CD4⁻, CD8⁻ regulatory T cell expresses at least one of the following markers CD3⁺, TCR β⁺, CD44⁺, CD69⁺, or CD28⁺, but not NK1.1. It is further desirable that the said CD4⁻, CD8⁻ regulatory T cell is Foxp3⁻. The converted CD4⁻, CD8⁻ regulatory T cell has the characteristics of suppressing an antigen-specific immune response to said antigen in a subject. Isolating said CD4⁻, CD8⁻ regulatory T cell is by selection of said cell expressing the cell surface marker CD3, and lacking the cell surface marker CD4. In a preferable embodiment of this embodiment of this invention, said isolating is done using at least one of an enrichment column, or cell sorting.

The invention also describes a method wherein said CD4⁻, CD8⁻ regulatory T cell is obtained by at least 4 rounds of antigen stimulation in the presence of at least one of the cytokines IL-2, or IL-15. In a preferable embodiment of the invention, said CD4⁻, CD8⁻ regulatory T cell can suppress at least one of proliferation, or activation of an antigen-specific responder T cell. The responder T cell population subject to suppression may express the phenotype CD4⁺, CD25⁻ or CD4⁺, CD25⁺. The responder T cell population subject to suppression may also be CD8⁺. The antigen used in generation of said CD4⁻, CD8⁻ regulatory T cell can be an auto-, allo-, or xenoantigen. The source of said antigen can be from CD3⁻ mature bone marrow dendritic cells or antigen presenting cells.

The invention also describes a method wherein disappearance of the cell surface CD4 molecule on a converted CD4⁻, CD8⁻ T cell, was a result of CD4 gene silencing. In a preferred embodiment of the invention, the CD4⁻, CD8⁻ regulatory T cell is converted from a CD4⁺CD25⁻ T cell. In another preferred embodiment of the invention, the CD4⁻, CD8⁻ regulatory T cell is converted from a CD4⁺CD25⁺ T cell. In a further preferred embodiment of this invention, said converted CD4⁻, CD8⁻ regulatory T cell is expanded by at least one of the cytokines IL-2, or IL-15.

The invention also features a method for inhibiting, treating or modulating an antigen specific immune response in a subject in need thereof, wherein said method comprising of administering said isolated CD4⁻, CD8⁻ T regulatory cell. In one preferred embodiment, an antigen-specific immune response may include graft rejection, an autoimmune disorder, graft versus host disease (GVHD), a response to a tumor cell, a response to an infection, or a response to an allergen.

In a further embodiment of the invention, said inhibition, treatment or modulation of an antigen-specific immune response is by augmenting activated induced cell death (AICD) of naïve or activated responder T cells. Said responder T cells preferably have the phenotype CD4⁺, CD25⁻ or CD4⁺, CD25⁺, or CD8⁺ and said AICD is by apoptosis of said responder. Preferably, AICD-induced apoptosis of said responder T cell is partially dependent on perforin. In another embodiment, AICD-induced apoptosis of said responder T cell is partially dependent on granzyme B.

### EXAMPLES

The following examples are intended to illustrate the invention. They are not meant to limit the invention in any way.

### Example 1

### Peripheral CD4⁺ T Cells Convert to CD4⁻ Cells

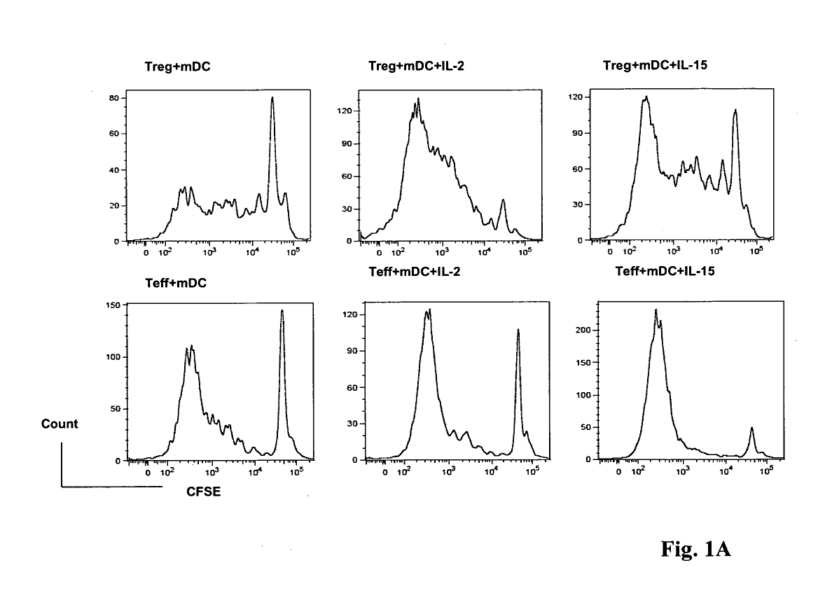

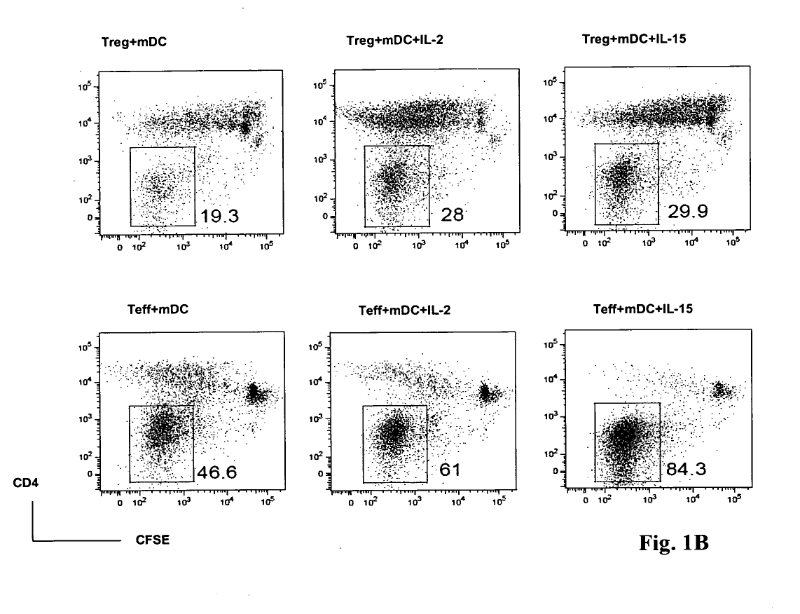

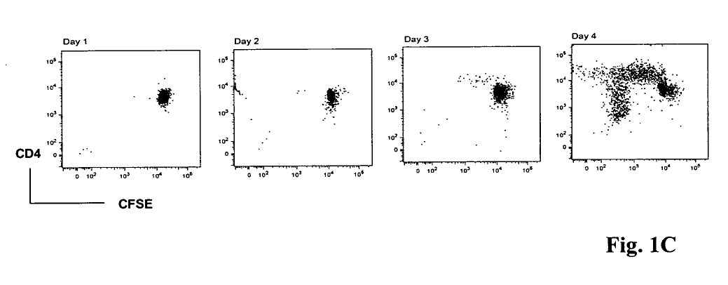

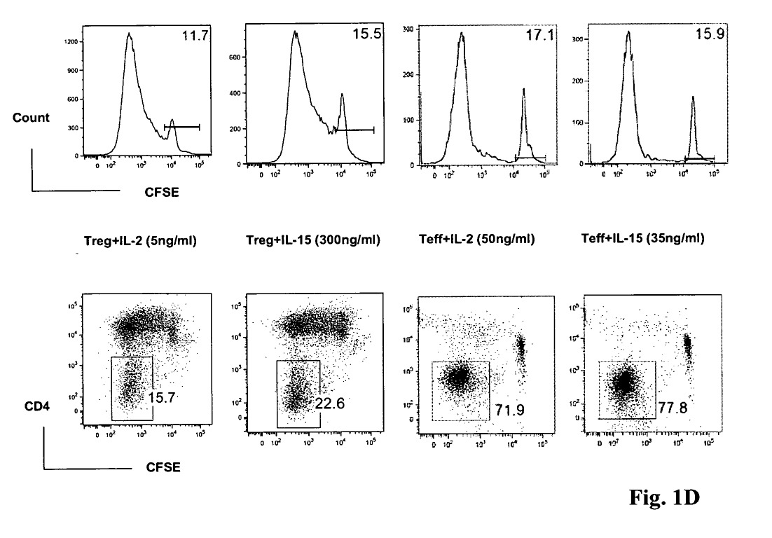

Mature bone marrow-derived dendritic cells (BM DC) have shown the ability to trigger vigorous proliferation of allogeneic CD4⁺CD25⁺ T regulatory cells (Treg) in vitro. In an attempt to study the effects of T cell growth factors (TCGFs) on the activation and proliferation of CD4⁺CD25⁺ Treg and CD4⁺CD25⁻ T cells in vitro, we employed LPS mature allogeneic BM DC with or without TCGFs in a mixed lymphocyte reaction (MLR). Fluorescent CFSE labeled C57BL/6 CD4⁺CD25⁺ and CD4⁺CD25⁻ T cells co-cultured with mature allogeneic DC proliferated vigorously in 6 day MLR, and the addition of rIL-2, or rIL-15 further enhanced the proliferation (FIG. 1A). Interestingly, we found that a significant proportion of proliferated cells were CD4 negative. The percentage of CD4 negative cells ranged from 19.3%, when CD4⁺CD25⁺ T cells were cultured with mature DC, to 84.3%, when CD4⁺CD25⁻ T cells were cultured with mature DC plus rIL-15 (FIG. 1B).

To examine the sources of these CD4⁻ cells, highly purified CD4⁺CD25⁻ T cells (>99%) were cultured with mature DC plus rIL-15. The CD4⁻ cells were not detectable at day 1, 2, and 3 of MLR, indicating that the CD4⁻ cells were not from the possible contamination from the culture. The CD4⁻ cells appeared at day 4 of MLR accompanied by robust cell proliferation, indicating that the CD4⁻ cells were converted from proliferated CD4⁺ T cells. The CFSE fluorescent intensity of proliferated T cells indicated that the conversion of CD4⁺CD25⁻ T cells to CD4⁻ cells took place only after 4-5 rounds of alloantigen triggered CD4⁺ T cell proliferation (FIG. 1C).

To quantitatively evaluate the impact of rIL-2 and rIL-15 on the conversion of CD4⁺ T cells to CD4⁻ cells, we titrated the doses of rIL-2 and rIL-15 in the MLR. Significant differences of enhancement efficacy between rIL-2 and rIL-15 alloantigen triggered proliferation of CD4⁺CD25⁺ versus CD4⁺CD25⁻ T cells were noted (FIG. 1D). However, we found that at an equivalent concentration in terms of supporting alloantigen triggered either CD4⁺CD25⁻ or CD4⁺CD25⁺ T cell proliferation, measured by approximately 85% of divided CD4⁺CD25 and CD4⁺CD25⁺ T cells. Within this gated population, rIL-2 and rIL15 exerted similar effects to enhance the conversion of approximately 70% of CD4⁺CD25⁻ or 20% of CD4⁺CD25⁺ T cells into CD4⁻ cells (FIG. 1D).

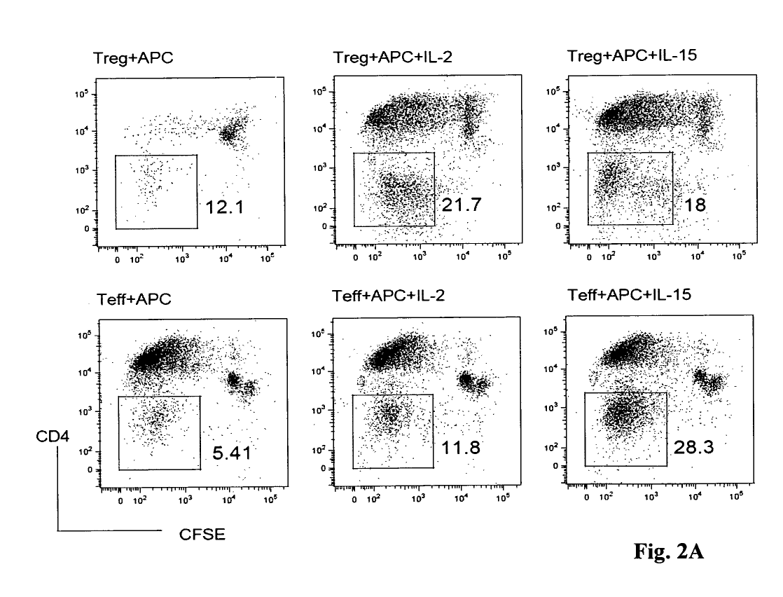

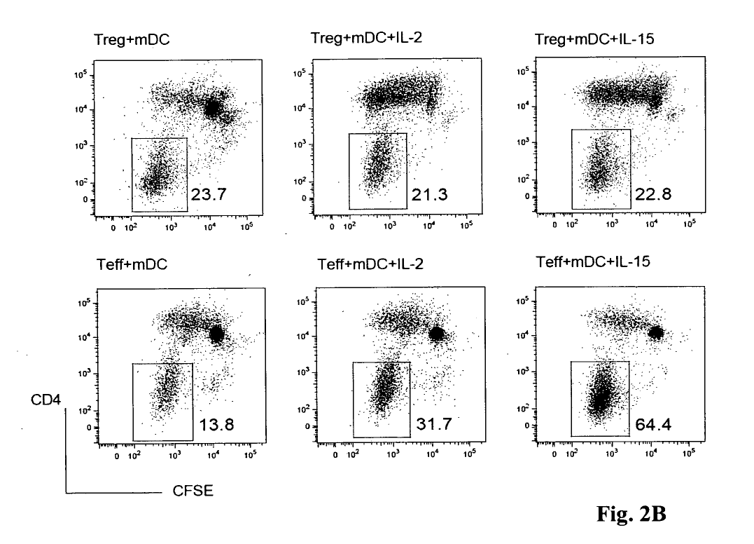

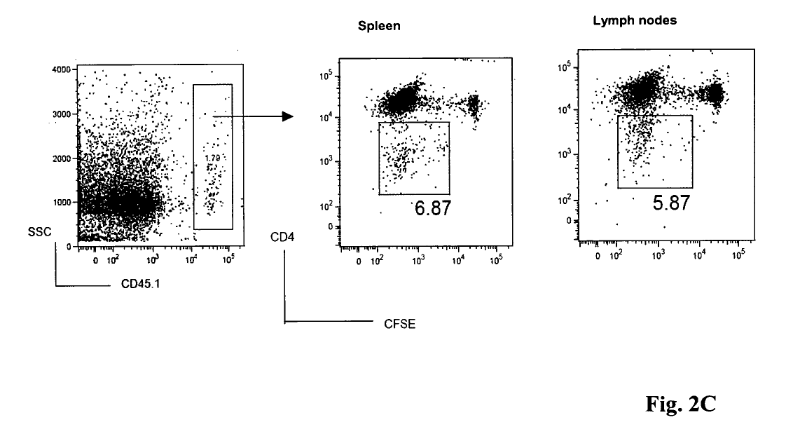

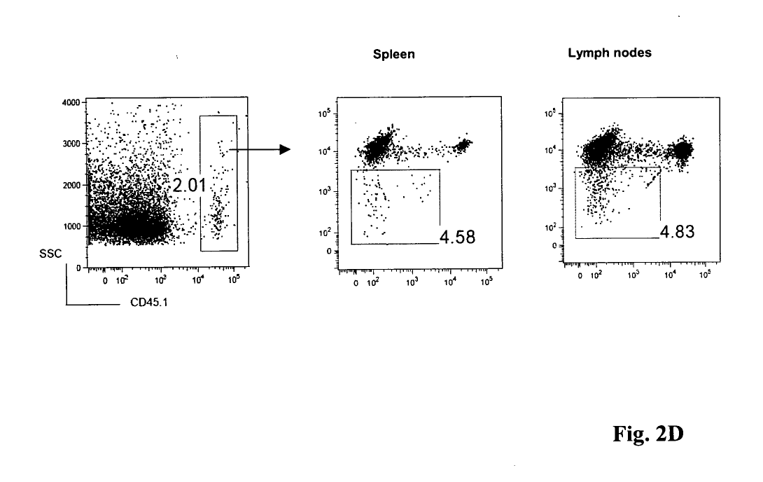

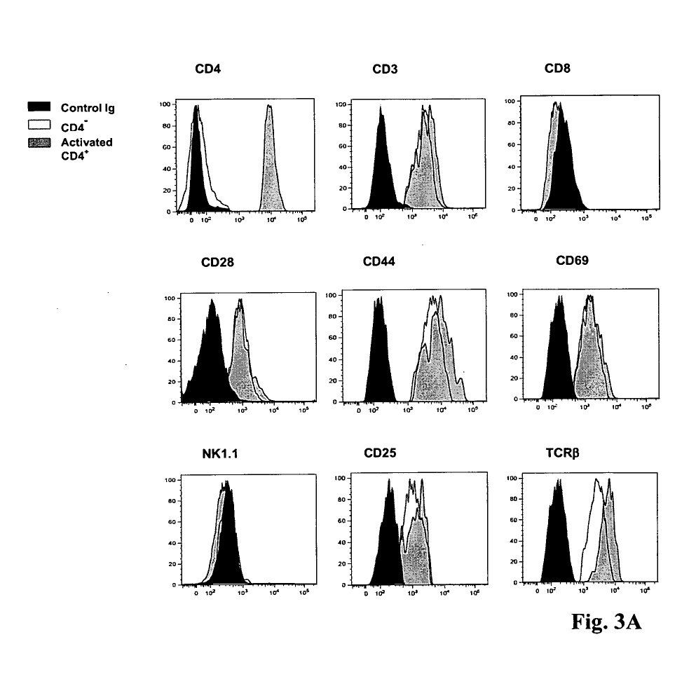

We further examine the conversion of CD4⁺ T cells to CD4⁻ T cells in vitro in a MLR by using mitomycin C treated DBA/2 allogeneic APC or syngeneic mature DC. Both CFSE labeled CD4⁺CD25⁻ and CD4⁺CD25⁺ T cells from spleen and lymph nodes of naïve congenic CD45.1 C57BL/6 mice can be converted to CD4⁻ cells after 6-day stimulation with mitomycin C treated DBA/2 allogeneic APC (FIG. 2A) or syngeneic mature DC (FIG. 2B) in vitro. The addition of rIL-2 or rIL-15 in the culture significantly enhanced the conversion (FIG. 2A). Moreover, to track the CD4 expression during alloantigen triggered or homeostatic proliferation in vivo, we adoptively transferred CFSE labeled highly purified CD4⁺ T cells (>99%) from congenic CD45.1 naïve C57BL/6 mice into allogeneic B6D2F1 or syngeneic immune deficient Rag KO mice. As FIG. 2C shows, the congenic CD45.1⁺CD4⁺ T cells underwent a robust proliferation in allogeneic B6D2F1 hosts 3 days after adoptive transfer. After 4-5 rounds of proliferation, 5.87% and 6.87% of CD45.1⁺ CD4⁺ T cells harvested from lymph nodes and spleen of B6D2F1 hosts converted to CD4⁻ T cells respectively (FIG. 2C). A similar pattern of in vivo homeostatic proliferation and conversion of the congenic CD45.1⁺CD4⁺ T cells harvested from syngeneic Rag KO hosts, 7 days after adoptive transfer, was demonstrated in FIG. 2D.

### Example 2

### Converted CD4⁻ Cells have a Unique Phenotype and Gene Expression Profile

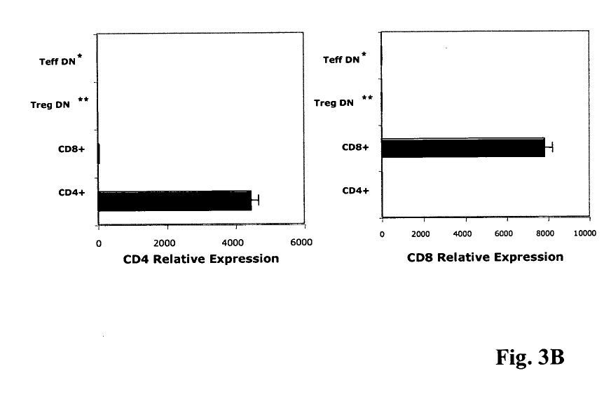

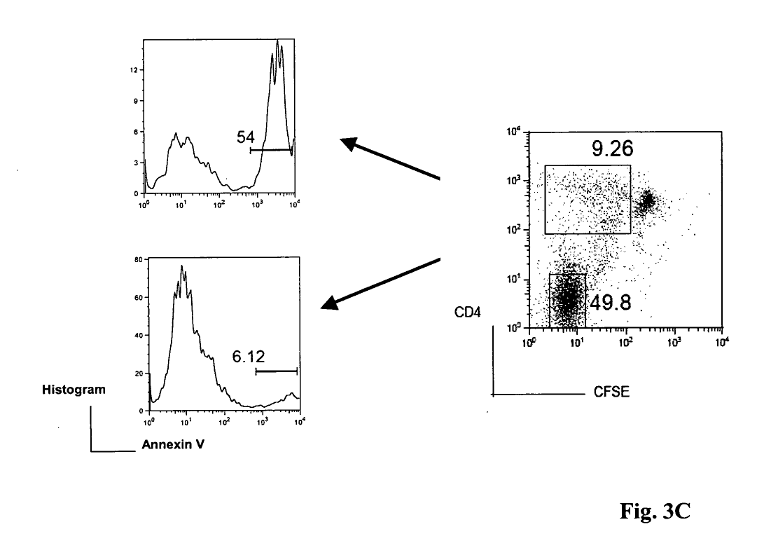

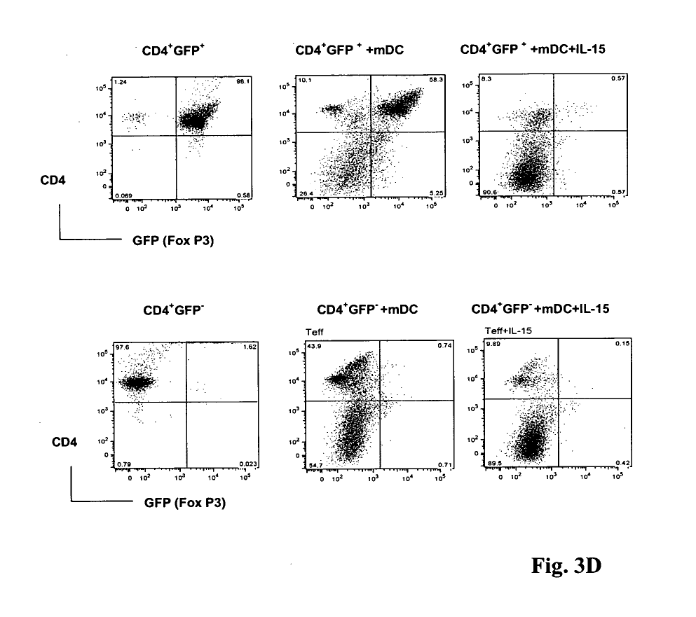

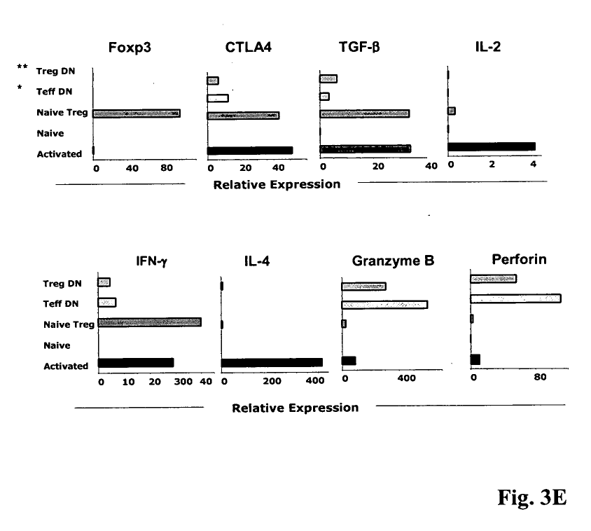

To characterize the converted CD4⁻ cells, we examined the expression of cell surface markers. The converted CD4⁻ cells express a unique set of cell surface markers, as shown in FIG. 3A. The CD4⁻ T cells are CD4⁻, CD8⁻, CD3⁺, TCR β⁺, NK1.1⁻, CD44⁺, CD25⁺, CD69⁺, CD28⁺. Since the converted CD4⁻ cells are CD8⁻ and CD3⁺, we have named them CD4⁺ converted double negative (DN) T cells.

To determine the mechanism of the disappearance of CD4 expression on cell surface, we analyze the CD4 gene expression of converted CD4⁻ T cells by using real-time PCR. As shown in FIG. 3B, the CD4 gene was highly expressed in CD4⁺ T cells. In contrast, there was no detectable CD4 gene expression in converted CD4⁻ T cells. In addition, the CD8 gene was highly expressed in CD8⁺ T cells, but not in CD4⁺ and converted DN T cells. Thus, the CD4 gene was silent in converted DN T cells.

Previous studies reported that the activation induced cell death (AICD) is a routine consequence of T cell activation and apoptotic events occurring after a discrete number of T cell divisions. We investigated the apoptotic events between the proliferated CD4⁺ and DN T cell subsets. There were 54% Annexin V⁺ staining T cells among the proliferated CD4⁺ T cells after 5 days in vitro MLR (FIG. 3C). Surprisingly, there were only 6.12% Annexin V⁺ staining positive cells among converted DN T cells, even though they had gone through 4-8 rounds of cell division (FIG. 3C) indicating that the converted DN T cells were resistant to AICD.

The forkhead family transcription factor Foxp3 acts as the Treg cell lineage specification factor and thus identifies Treg cells independently of CD25 expression. Using a gene-targeting approach described previously, Foxp3^{gfp} knock-in mice were generated in which a bicistronic EGFP reporter was introduced into the endogenous Foxp3 locus (Bettelli et al., Nature 441 (7090):235-8 (2006)). By using CD4⁺CD25⁻Foxp3^{gfP+} and CD4⁺CD25⁺Foxp3^{gfp-} T cells from Foxp3^{gfp-} knock-in C57BL/6 mice, we found that CD4⁺CD25⁺Foxp3^{gfp+} T cells lost their Foxp3^{gfp} expression when they switched to DN T cells and CD4⁺CD25⁻ Foxp3^{gfp-} T cells remained Foxp3^{gfp} negative when they switched to DN T cells (FIG. 3D). In summary, the converted DN T cells from both CD4⁺CD25⁻ and CD4⁺CD25⁺ origin were Foxp3 negative.

A quantitative real-time PCR technique was used to analyze the gene expression profile of converted DN T cells. As shown in FIG. 3D, DN T cells converted from both CD4⁺CD25⁻ and CD4⁺CD25⁺ T cells did not express Foxp3, and expressed other Treg related CTLA-4 and TGFβ genes at low levels (FIG. 3E). IL-2, IL-4 and IFNγ genes that were highly expressed by activated CD4⁺ T cells, were expressed at low levels by DN T cells. Interestingly, DN T cells expressed high levels of the cytotoxic lymphocyte related genes perforin and granzyme B. Thus, DN T cells converted from both CD4⁺CD25⁻ or CD4⁺CD25⁺ T cells shared similar gene expression profile that was distinctive from naïve CD4⁺CD25⁻, naïve CD4⁺CD25⁺ Treg, and activated CD4⁺ T cells.

### Example 3

### Converted DN T Cells are Regulatory T Cells

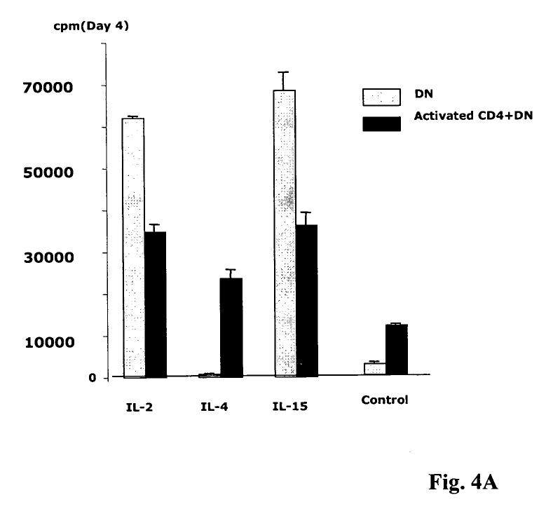

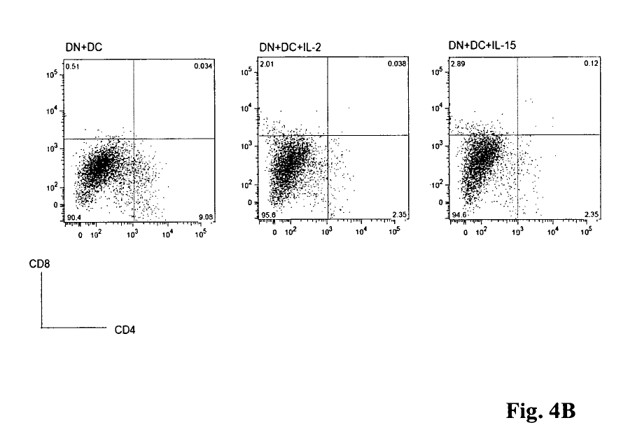

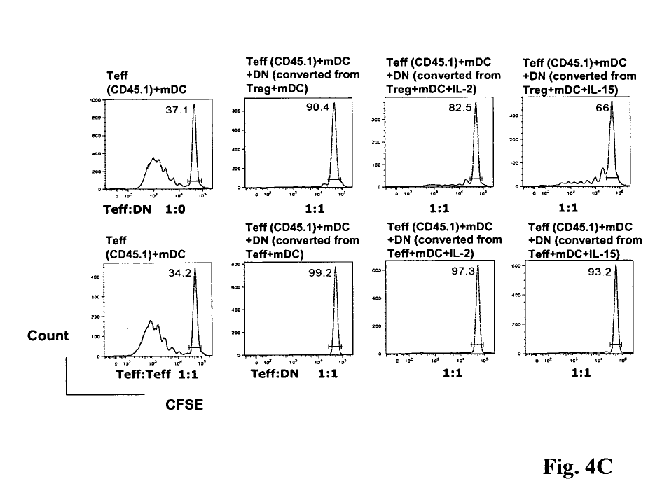

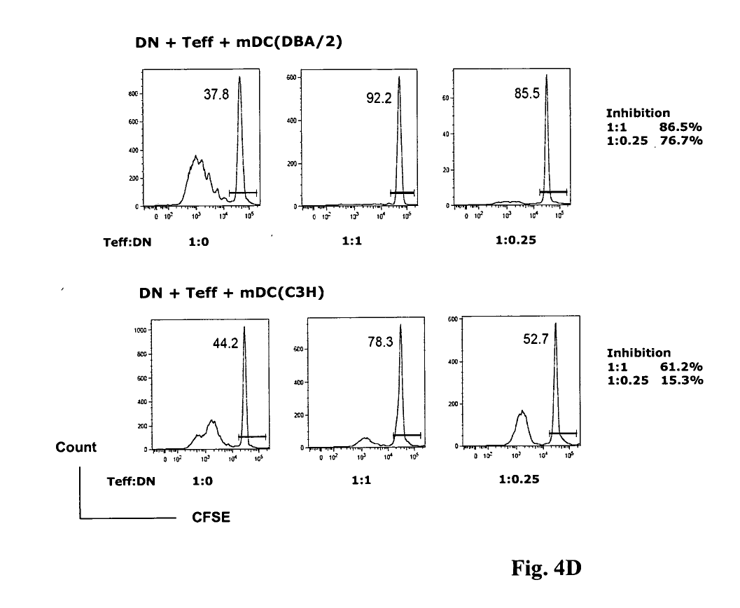

To analyze the functional properties of CD4⁺ converted DN T cells, we isolated CD4⁺ and DN T cells from primary MLR by cell sorting. Upon restimulation with same strain of mature DC (DBA/2) as used in primary MLR, C57BL/6 CD4⁺ T cells proliferated vigorously, and the addition of rIL-2, rIL-4 or rIL-15 further enhanced proliferation (FIG. 4A). In contrast, DN T cells were hyporesponsive when restimulated by mature DC. Interestingly, rIL-2 or rIL-15, but not rIL-4, completely restored the responsiveness of DN T cells (FIG. 4A). Moreover, DN T cells retained a stable phenotype after re-stimulation with mature DC, even after robust proliferation by restimulation with mature DC plus IL-2 or IL-15 (FIG. 4B).

We further analyzed the effects of DN T cells on alloantigen-triggered proliferation of naïve CD4⁺CD25⁻ T cells. As shown in FIG. 4C, CFSE labeled naïve congenic CD45.1 C57BL/6 CD4⁺CD25⁻ T cells underwent vigorous proliferation during 5 day MLR with either allogeneic DBA/2 or C3H derived mature DC (FIG. 4C, and FIG. 4D). The DN T cells converted from both naïve C57BL/6 CD4⁺CD25⁻ and CD4⁺CD25⁺ T cells in a primary MLR with DBA/2 mature DC exerted a powerful inhibition of same alloantigen triggered proliferation of naïve CD4⁺CD25⁻ T effectors when co-cultured in a mixture of DN T cells with T effectors at 1:1 ratio. Interestingly, the addition of rIL-2 and rIL-15 in a primary culture, which significantly enhanced the conversion of CD4⁺ to DN T cells, did not have adverse effects on the potency of DN T cells to suppress alloantigen triggered naïve CD4⁺CD25⁻ T cell proliferation in a secondary MLR (FIG. 4C). Moreover, the potency of suppression of DN T cells tended to be alloantigen specific. As shown in FIG. 4D, DN T cells induced by DBA/2 alloantigen stimulation suppressed C3H alloantigen triggered naïve T-effector proliferation in a secondary MLR with lower efficacy. The differences of efficacy were more profound when T effectors were co-cultured with DN T cells at a 1:0.25 ratio (100,000 T effectors: 25,000 DN T cells, FIG. 4D).

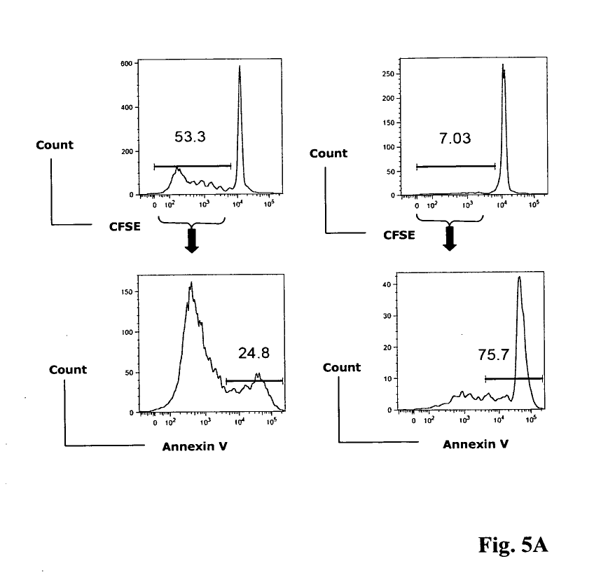

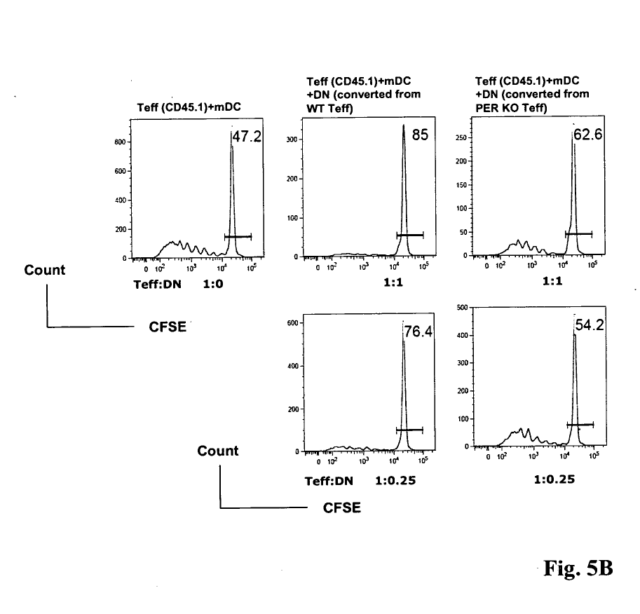

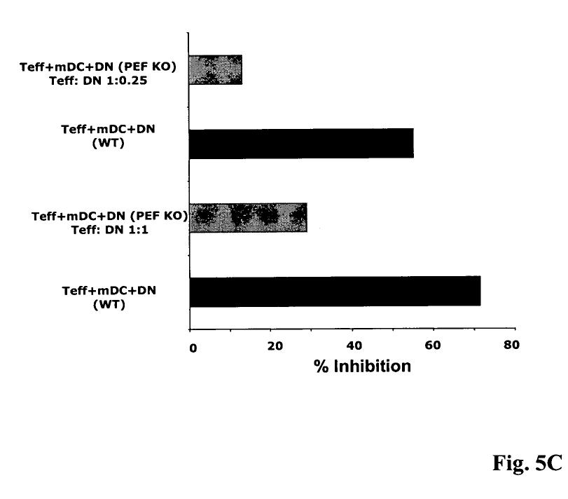

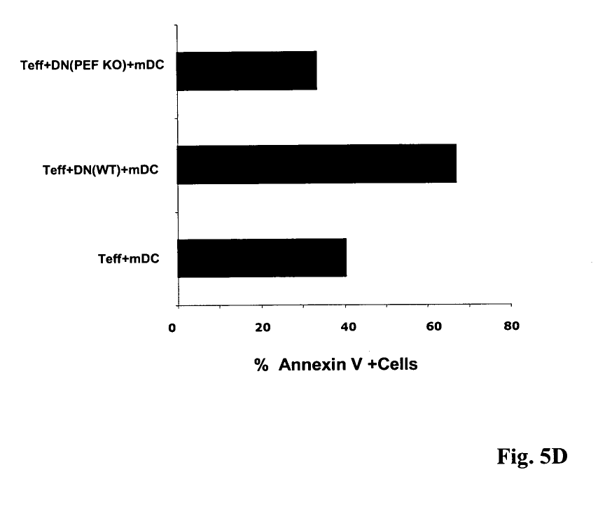

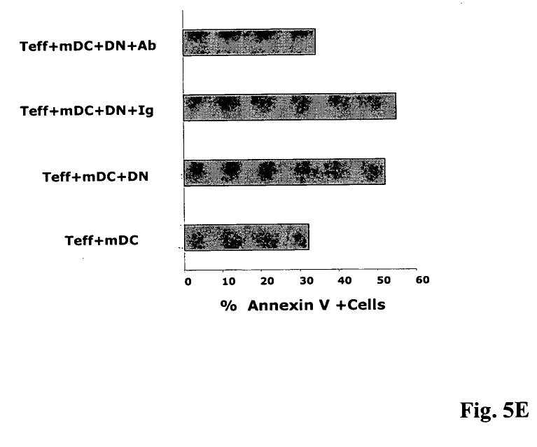

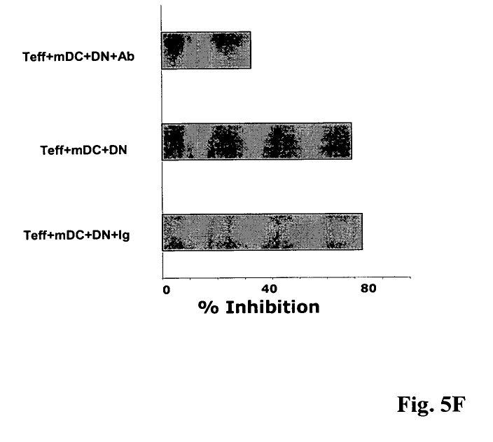

Activation induced cell death (AICD) is an important intrinsic mechanism that controls the magnitude of immune responses. We sought to determine the impact of DN T cells on AICD of proliferated CD4⁺ T cells by analyzing the apoptotic events among the proliferated CD4⁺ T cells co-cultured with mature DC with or without DN T cells. As shown in FIG. 5A, there was 24.8% of Annexin V⁺ cells among proliferated CD4⁺ T cells when CD4⁺CD25⁻ T cells were co-cultured with mature allogeneic DC alone. In contrast, there was 75.7% of Annexin V⁺cells among proliferated CD4⁺ T cells when CD4⁺CD25⁻ T cells co-cultured with mature allogeneic DC plus DN T cells at a 1:1 ratio (T effector: DN T cell). Thus, DN T cells exaggerated cell death of proliferated CD4⁺ T cells in mature DC stimulated MLR.

Perforin, a cytotoxic lymphocyte related cytokine, was highly expressed by DN T cells (FIG. 3D). To explore the mechanisms by which DN T cells suppress CD4⁺CD25⁻ proliferation, we examined the role of perforin in DN T cell mediated suppression. We compared the suppressive function of DN T cells converted from wild type C57BL/6 mice with that from perforin gene knock out C57BL/6 mice. The potency of DN T cells derived from perforin KO mice to suppress mature DC triggered proliferation of naïve CD4⁺CD25⁻ T effectors was significantly lower than that of wild type mice (FIGS. 5B and 5C). The inhibition rate was decreased from 71.6% to 29.2% when T effectors were co-cultured with DN T cells at a 1:1 ratio and from 55.3% to 13.3% when T effectors were co-cultured with DN T cells at a 1:0.25 ratio (FIG. 5C). FIG. 5D demonstrated that the addition of DN T cells derived from perforin KO mice, but not from wild type mice, in the co-culture did not increase Annexin V⁺ cells among activated T effectors, indicating that perforin played a role, at least in part, in DN T cell mediated cell death and suppression. We further showed that Granzyme B plays a role in DN T cell mediated suppression. Blockade of granzyme B by specific antibody decreased the Annexin V⁺ cells among activated T effectors (FIG. 5E), which is expressed as decrease in the percentage of inhibition of activated T effectors (FIG. 5F).

### Example 4

### Converted DN Modulatory T Cells can Suppress Alloimmune Responses In Vivo

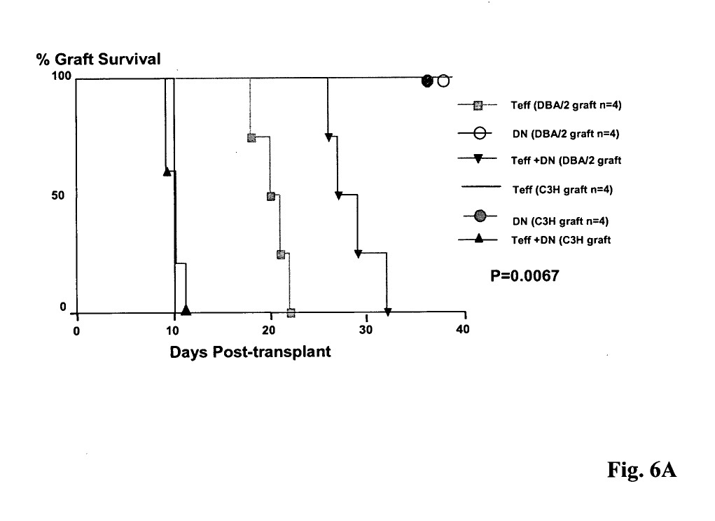

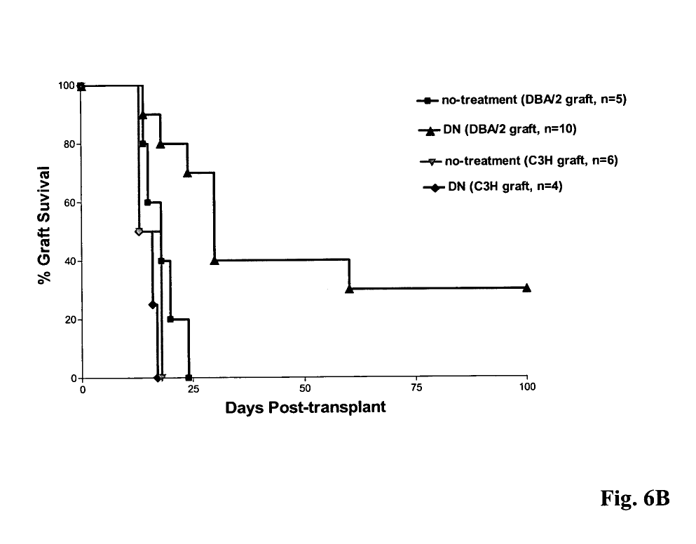

To further test the functional potential of the converted DN T cells in vivo, we utilized an adoptive transfer model of skin allografts described previously (Sanchez-Fueyo et al., J Immunol. 168:2274-2281 (2002)). C57BL/6 (H-2^{b}) RAG (⁻/⁻) recipients received 100,000 naïve C57BL/6 effector T cells with or without 100,000 DN T cells, which were converted from CD4⁺CD25⁻ T cells of naïve C57BL/6 mice by co-culture with mature DBA/2 (H-2^{d}) DC plus rIL-15 in MLR for 6 days. An alloantigen specific DBA/2 or control third party strain C3H (H-2^{k}) tail skin graft was placed the following day. As shown in FIG. 6A, adoptive transfer of 100,000 naïve C57BL/6 CD4⁺CD25⁻ T cells were capable of triggering acute rejection of DBA/2 or C3H skin allografts at mean graft survival time of 20 days and 10 days respectively. In contrast, adoptive transfer of same number of converted C57BL/6 DN T cells did not trigger rejection of DBA/2 or C3H skin allografts, indicating that the DN T cells were anergic upon alloantigen restimulation. However, significant prolongation of DBA/2 skin allografts occurred when an equal number of naïve C57BL/6 CD4⁺CD25 and DN T cells, converted from naïve C57BL/6 CD4⁺CD25⁻ T cells after 6 day MLR with DBA/2 mature DC plus IL-15, were co-transferred (FIG. 6A, p=0.0067). In contrast, the co-transferred of DN T cells did not protect third party strain C3H skin allografts from acute rejection (FIG. 6A). Thus DN T cells were capable of suppressing naïve CD4⁺CD25⁻ T cell triggered skin allograft rejection in vivo in an alloantigen specific manner.

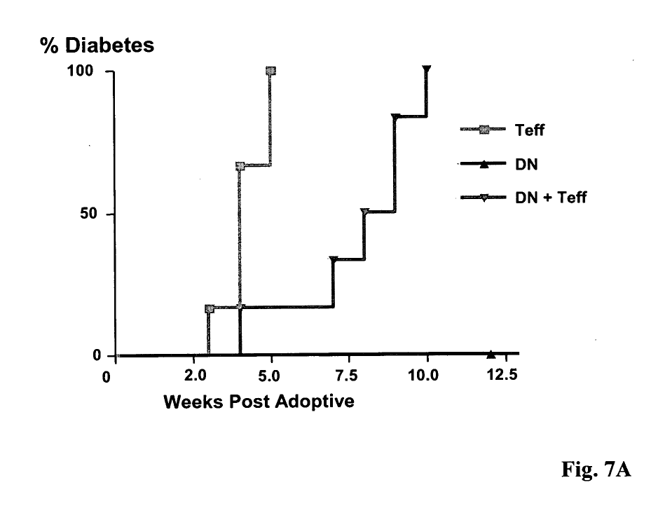

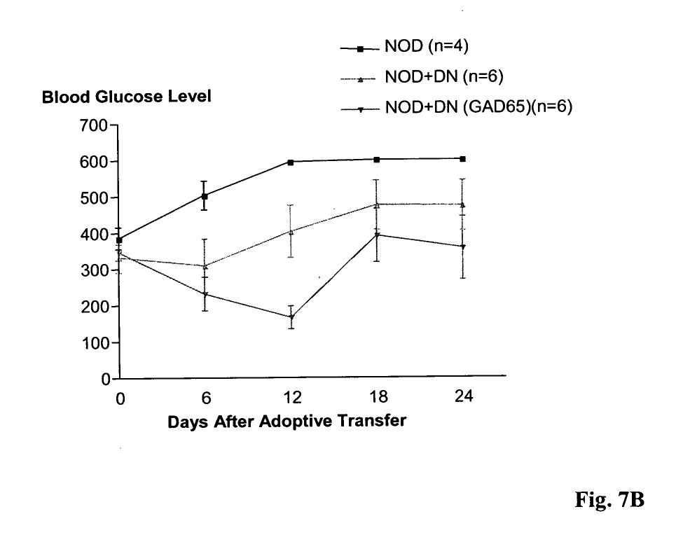

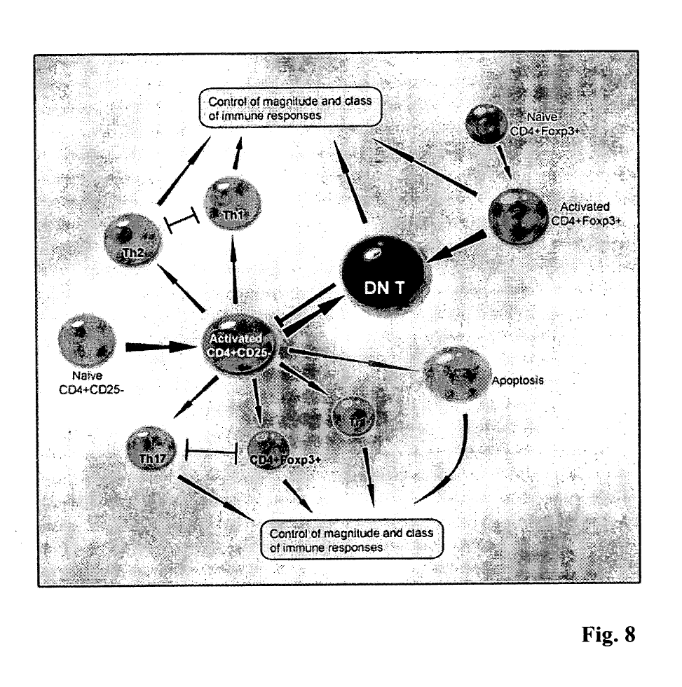

Based on the finding of allospecific suppressive function of DN T cells in the in vivo adoptive T cell transfer model of skin allograft, we sought to determine if the administration of a relatively small number of DN T cells as a monotherapy would have any effect on graft survival in an immunocompetent MHC completely mismatched transplant model. We chose a pancreatic islet transplant model in which 13×10⁶ DN T cells, converted from CD4⁺CD25⁻ T cells of naïve C57BL/6 mice by co-culture with mature DBA/2 (H-2^{d}) DC plus rIL-15 in MLR for 6 days, were transferred into streptozotocin induced diabetic C57BL/6 recipients at the time of islet cell transplantation. As shown in FIG. 6B, the transfer of 13×10⁶ DN T cells resulted in a statistically significant prolongation of alloantigen specific DBA/2 strain, but not third party C3H strain, islet allograft survival in comparison with that of untreated control group (p=0.005). This confirms the utility of the ex vivo CD4⁺ T cell converted alloantigen specific DN regulatory T cells as an immune modulatory therapy in preventing allograft rejection in a MHC mismatched islet allograft model. We further show that DN regulatory T cells can prevent the development of autoimmune diabetes. As show in FIG. 7A DN T cells protect NOD/SCID mice from autoimmune diabetes induced by diabetogenic T cells from diabetic NOD mice. The diabetes of NOD/SCID mice was induced by T cells from diabetic NOD mice. Co-injection of NOD DN T cells significantly protected the mice from diabetes. Furthermore, we show that this is an antigen-specific protection as islet GAD65 antigen-specific DN T cells were more potent than antigen-nonspecific DN T cells in blocking autoimmune diabetes in new onset diabetic NOD mice (FIG. 7B).

### Other Embodiments

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What is claimed is:

1. An isolated regulatory T cell, said cell having the phenotype CD4⁻, CD8⁻, said cell expressing at least one of CD44⁺, CD69⁺, or CD28⁺.

2. A regulatory T cell of claim 1, wherein said cell also expresses at least one of CD3⁺, CD25⁺, TCR β⁺.

3. A regulatory T cell of claim 1, wherein said cell has the phenotype NK1.1⁻.

4. A regulatory T cell of claim 1, wherein the cell has the phenotype Foxp3⁻.

5. A regulatory T cell of claim 1, wherein said cell expresses low levels of IL-2, IL-4, IFN-γ, CTLA-4, TGF-β, and high levels of perforin and granzyme B.

6. A regulatory T cell of claim 1, wherein the CD4⁻ phenotype of said cell is the result of CD4 gene silencing.

7. A regulatory T cell of claim 1, wherein said cell is more effective at suppressing antigen-specific proliferation of naïve CD4′, CD25⁻ T cells than said cell is at suppressing antigen-nonspecific proliferation of naïve CD4⁺, CD25⁻ T cells.

8. A method for obtaining a CD4⁻, CD8⁻ regulatory T cell, said method comprising of:
a) isolating a CD4⁺, CD8⁻ cell from a sample;
b) culturing said CD4⁺, CD8⁻ cell with antigen and at least one of IL-2, or IL-15;
c) isolating said CD4⁻, CD8⁻;
d) wherein said isolated CD4⁻, CD8⁻ cell has the characteristics of suppressing an antigen-specific immune response to said antigen in a subject.

9. The method of claim 8, wherein said isolated CD4⁺, CD8⁻ cell is CD25⁺.

10. The method of claim 8, wherein said isolated CD4⁺, CD8⁻ cell is CD25⁻.

11. The method of claim 8, wherein said isolated CD4⁻, CD8⁻ cell is Foxp3⁻.

12. The method of claim 8, wherein said CD4⁻, CD8⁻ cell obtained by said culturing step b) is at least four rounds of antigen stimulation.

13. The method of claim 8, wherein said CD4⁻, CD8⁻ cell suppresses at least one of proliferation, or activation of an antigen-specific responder T cell.

14. The method of claim 13, wherein said responder T cells are CD4⁺, CD25⁻ or CD4⁺, CD25⁺.

15. The method of claim 13 wherein said responder T cells are CD8⁺.

16. The method of claim 8, wherein said CD4⁻, CD8⁻ expresses the proteins perforin and granzyme B.

17. The method of claim 8, wherein said CD4⁻, CD8⁻ cell expresses at least one of the following markers CD3⁺, TCR β⁺, CD44⁺, CD69⁺, or CD28⁺.

18. The method of claim 8, wherein said CD4⁻, CD8⁻ cell has the phenotype NK1.1⁻.

19. The method of claim 8, wherein said antigen is an auto-, allo-, or xenoantigen.

20. The method of claim 8, wherein said antigen is present on CD3⁻ mature bone marrow dendritic cells or antigen presenting cells.

21. The method of claim 20, wherein said antigen presenting cells are B cells, monocytes, macrophages or dendritic cells.

22. The method of claim 8, wherein isolating of said CD4⁻, CD8⁻ cell is by selection of said cell expressing the cell surface marker CD3 and not expressing the cell surface marker CD4.

23. The method of claim 22, wherein said isolating is done using at least one of an enrichment column, or cell sorting.

24. The method of claim 8, wherein said CD4⁻, CD8⁻ cell is expanded by at least one of IL-2, or IL-15.

25. A method for inhibiting, treating, or modulating an antigen specific immune response in a subject in need thereof, wherein said method comprising of administering said CD4⁻, CD8⁻ cell of claim 8.

26. The method of claim 25, wherein said antigen-specific immune response is graft rejection, an autoimmune disorder, graft versus host disease (GVHD), a response to a tumor cell, a response to an infection, a response to an allergen.

27. The method of claim 25, wherein said inhibition, treatment, or modulation of an antigen-specific immune response is by augmenting activation induced cell death (AICD) of naïve or activated responder T cells.

28. (canceled)

29. (canceled)

30. The method of claim 27, wherein said AICD is by apoptosis of said responder cell.

31. The method of claim 30, wherein said AICD by apoptosis is partially dependent on perforin.

32. The method of claim 30, wherein said AICD by apoptosis is partially dependent on granzyme B.

33.-48. (canceled)
